(19) United States
(12) Patent Application Publication
(10) Pub. No.: US 2021/0060242 A1
(43) Pub. Date: Mar. 4, 2021

(54) BUBBLE DETECTOR AND RELATED BUBBLE DETECTION METHODOLOGY FOR A MEDICATION FLUID INFUSION SYSTEM

(71) Applicant: MEDTRONIC MINIMED, INC., Northridge, CA (US)

(72) Inventors: Davy Tong, Thousand Oaks, CA (US); Hsi C. Fusselman, Simi Valley, CA (US); Sarnath Chattaraj, Simi Valley, CA (US)

(21) Appl. No.: 16/552,975

(22) Filed: Aug. 27, 2019

(51) Int. Cl. A61M 5/168; A61M 5/142; A61M 5/36

(52) U.S. Cl. CPC A61M 5/16831; A61M 5/14248; A61M 5/16813; A61M 5/365; A61M 2205/3317; A61M 2205/52; A61M 2205/18; A61M 2205/502; A61M 2205/50; A61M 2205/8206; A61M 2205/332; A61M 2205/3592

(57) ABSTRACT

A bubble detector, a medical device system that includes the detector, and a related operating methodology are disclosed here. The detector is designed and operated to detect presence of bubbles in a fluid delivery tube. The detector includes a capacitive sensor, a control circuit to control operation of the sensor to measure capacitance associated with fluid in the fluid delivery tube, a power source to provide voltage across the capacitor electrodes and to provide operating power to the control circuit, a communication module to communicate sensor data from the detector, and a housing with structural features to secure the detector to the fluid delivery tube and to maintain the capacitor electrodes in position relative to the fluid delivery tube. The sensor data is indicative of the measured capacitance associated with the fluid in the fluid delivery tube.

### TECHNICAL FIELD

Embodiments of the subject matter described herein relate generally to fluid delivery devices and systems, such as a medication fluid infusion device. More particularly, embodiments of the subject matter relate to a bubble detector that uses a capacitive sensing technique to detect the presence of bubbles in a fluid delivery tube.

### BACKGROUND

The prior art includes a variety of fluid delivery systems that utilize fluid conduits to transport fluid from one location/device to another location/device. A medication fluid delivery system is designed to deliver medication fluid to a patient in a controlled and regulated manner. In this context, certain diseases or conditions may be treated, according to modern medical techniques, by delivering a medication fluid or other substance to the body of a patient, either in a continuous manner or at particular times or time intervals within an overall time period. For example, diabetes is commonly treated by delivering defined amounts of insulin to the patient at appropriate times. Some common modes of providing insulin therapy to a patient include delivery of insulin through manually operated syringes and insulin pens. Other modern systems employ programmable fluid infusion devices (e.g., continuous insulin infusion devices such as insulin pumps) to deliver controlled amounts of insulin or other drugs to a patient.

A medication fluid infusion device suitable for use as an insulin pump may be realized as an external device, e.g., a device designed for use in a generally stationary location (for example, in a hospital or clinic), or a device configured for ambulatory or portable use (to be carried by a patient). External fluid infusion devices may establish a fluid flow path from a fluid reservoir to the patient via, for example, a suitable hollow tubing. The hollow tubing may be connected to a hollow fluid delivery needle that is designed to pierce the patient's skin to deliver an infusion fluid to the body. Alternatively, the hollow tubing may be connected directly to the patient's body through a cannula or set of micro-needles.

It is desirable to detect and reduce the amount of air bubbles in a medication fluid before delivering the fluid to the patient. Small bubbles may be introduced into the medication fluid during a reservoir filling operation, for example, when the fluid reservoir is filled from a vial using a syringe. Bubbles can also be generated during temperature or altitude changes. Although patients are instructed to eliminate air from a filled reservoir, some bubbles may remain.

Accordingly, it is desirable to have a bubble detection component and related detection methodology that are designed to mitigate the effects of air bubbles within a medication fluid flow path. Furthermore, other desirable features and characteristics will become apparent from the subsequent detailed description and the appended claims, taken in conjunction with the accompanying drawings and the foregoing technical field and background.

### BRIEF SUMMARY

A medical device system is disclosed here. An exemplary embodiment of the system includes a medication fluid infusion device having a fluid reservoir. The medication fluid infusion device is operable to regulate and control delivery of a medication fluid from the fluid reservoir. The system also includes an infusion set having a fluid delivery tube and an infusion site component fluidly and mechanically coupled to a downstream end of the fluid delivery tube. The infusion set is couplable to the medication fluid infusion device to establish a fluid connection with the fluid reservoir. The system also includes a bubble detector that is couplable to the fluid delivery tube of the infusion set. An embodiment of the bubble detector includes: a capacitive sensor; a power source to provide operating voltage to the capacitive sensor; a control circuit to control operation of the capacitive sensor to measure capacitance associated with fluid in the fluid delivery tube; and a communication module to communicate sensor data from the bubble detector, the sensor data indicative of the measured capacitance associated with the fluid in the fluid delivery tube. The system also includes a controller to receive and analyze the sensor data from the bubble detector, and to determine presence of one or more bubbles in the fluid delivery tube based on analysis of the sensor data.

Also disclosed here is a bubble detector for detecting presence of one or more bubbles in a fluid delivery tube. An exemplary embodiment of the bubble detector includes: a capacitive sensor having capacitor electrodes; a control circuit to control operation of the capacitive sensor to measure capacitance associated with fluid in the fluid delivery tube; a power source to provide operating voltage across the capacitor electrodes and to provide operating power to the control circuit; a communication module to communicate sensor data from the bubble detector, the sensor data indicative of the measured capacitance associated with the fluid in the fluid delivery tube; and a housing for at least the capacitive sensor. The housing includes structural features to secure the bubble detector to the fluid delivery tube and to maintain the capacitor electrodes in position relative to the fluid delivery tube.

Also disclosed here is a method of detecting presence of one or more bubbles in a fluid delivery tube. An exemplary embodiment of the method involves: receiving sensor data from a capacitive sensor coupled to the fluid delivery tube, the sensor data indicative of measured capacitance associated with the fluid in the fluid delivery tube; analyzing the sensor data received from the capacitive sensor; determining a bubble-free state of the fluid in the fluid delivery tube when the analyzed sensor data indicates measured capacitance having a first detectable characteristic; determining presence of one or more bubbles in the fluid delivery tube when the analyzed sensor data indicates measured capacitance having a second detectable characteristic that is distinguishable from the first detectable characteristic; and initiating a corrective action in response to determining the presence of one or more bubbles in the fluid delivery tube.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a schematic plan view of an exemplary embodiment of a medical device system;

FIG. 2 is a schematic block diagram representation of an embodiment of a fluid infusion device that is suitable for use in the medical device system shown in FIG. 1;

FIG. 3 is a schematic block diagram representation of an embodiment of a bubble detector that is suitable for use in the medical device system shown in FIG. 1;

FIG. 4 is a schematic side view of a capacitive sensor and a fluid delivery tube;

FIG. 5 is a perspective view of semi-cylindrical electrodes of a capacitive sensor; and

FIG. 6 is a cross sectional view of a capacitive sensor and a fluid delivery tube.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

The subject matter described here relates to a medical device system that includes a fluid infusion device of the type used to treat a medical condition of a patient. The infusion device is used for infusing medication fluid into the body of a user. The non-limiting example described below relates to a medical device used to treat diabetes (for example, an insulin pump or other type of insulin infusion device), although embodiments of the disclosed subject matter are not so limited. Accordingly, the infused fluid is insulin in certain embodiments. In alternative embodiments, however, many other fluids may be administered through infusion such as, but not limited to, disease treatments, drugs to treat pulmonary hypertension, iron chelation drugs, pain medications, anti-cancer treatments, medications, vitamins, hormones, or the like.

For the sake of brevity, conventional features and technologies related to infusion system operation, insulin pump and/or infusion set operation, blood glucose sensing and monitoring, sensor signal processing, and other functional aspects of the fluid infusion system (and the individual operating components of the system) may not be described in detail here. Examples of infusion pumps used to administer insulin and other medications may be of the type described in, but not limited to, U.S. Pat. Nos. 6,485,465; 6,554,798; 6,558,351; 6,752,787; 6,932,584; and 7,621,893; which are herein incorporated by reference.

In accordance with certain exemplary embodiments described herein, a fluid infusion system utilizes an infusion set that functions to deliver a medication fluid to the body of a patient via a fluid delivery tube and an infusion site component that is designed for attachment to the body of the patient. The disclosed system also includes a suitably configured bubble detector component that can be removably coupled to the fluid delivery tube of the infusion set for purposes of detecting the presence of one or more bubbles in the fluid delivery tube. The bubble detector employs a capacitive sensor that functions to measure the capacitance of the fluid delivery tube and the fluid passing through the tube in an ongoing manner (e.g., during fluid delivery operations). The output of the capacitive sensor is monitored and analyzed to determine whether the fluid in the tube is free of bubbles or whether one or more bubbles are present in the tube.

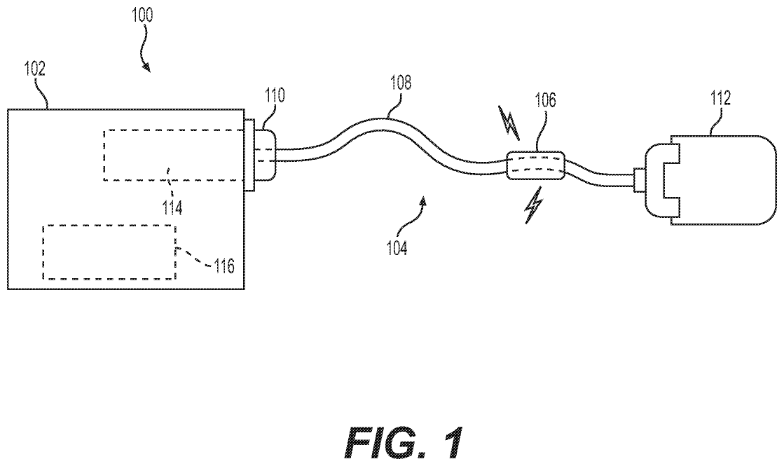

Referring now to the drawings, FIG. 1 is a schematic plan view of an exemplary embodiment of a medical device system 100. The system 100 includes at least the following hardware components: a medication fluid infusion device 102 (e.g., an insulin pump); an infusion set 104; and a bubble detector 106. FIG. 1 shows the system 100 in its assembled state—the infusion set 104 is physically and fluidly coupled to the fluid infusion device 102, and the bubble detector is attached to a fluid delivery tube 108 of the infusion set 104. The illustrated embodiment of the infusion set 104 includes, without limitation: the fluid delivery tube 108; a reservoir connector 110 at the upstream end of the tube 108; and an infusion site component 112 that is fluidly and mechanically coupled to the downstream end of the tube 108. The reservoir connector 110 can be physically coupled to the fluid infusion device 102 to establish a fluid connection with a fluid reservoir 114 of the fluid infusion device 102. For the configuration shown in FIG. 1, the infusion set 104 is couplable to the fluid infusion device 102 to establish a fluid connection with the fluid reservoir 114, and the fluid infusion device 102 is operable to regulate and control delivery of a medication fluid from the fluid reservoir 114, through the fluid delivery tube 108, and into the body of the patient via the infusion site component 112. The fluid infusion device 102 is realized as an electronic computer-based or processor-based device that includes at least one controller 116 that is suitably programmed to regulate and control the operation of the fluid infusion device 102 as needed. In accordance with the embodiment shown in FIG. 1, the controller 116 is implemented as an integral component of the fluid infusion device 102.

The fluid infusion device 102 is designed to be a patient-carried or patient-worn component, and the patient-worn infusion set 104 cooperates with the fluid infusion device 102 to deliver medication fluid to the body of the patient via the tube 108. The fluid infusion device 102 may leverage a number of conventional features, components, elements, and characteristics of existing fluid infusion devices. For example, the fluid infusion device 102 may incorporate some of the features, components, elements, and/or characteristics described in U.S. Pat. Nos. 6,485,465 and 7,621,893, the relevant content of which is incorporated by reference herein.

The fluid infusion device 102 accommodates the fluid reservoir 114 for the medication fluid that is delivered to the user. The infusion set 104 defines the fluid flow path from the fluid reservoir 114 to the body of the patient. When assembled as depicted in FIG. 1, the tube 108 extends from the fluid infusion device 102 to the infusion site component 112, which in turn provides a fluid pathway to the body of the patient via a subcutaneous conduit. For the illustrated embodiment, the reservoir connector 110 is realized as a removable cap or fitting that is suitably sized and configured to accommodate replacement of fluid reservoirs (which are typically disposable) as needed. In this regard, the reservoir connector 110 is designed to accommodate the fluid path from the fluid reservoir 114 to the tube 108.

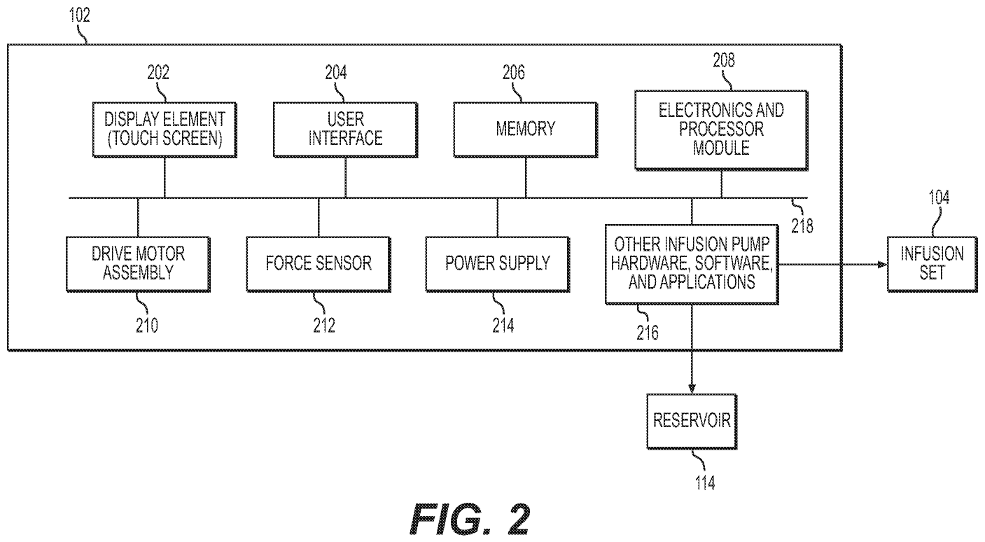

In practice, the fluid infusion device 102 includes an electronics module, processing logic, software applications, and/or other features that are used to carry out the various operating processes described here. In this regard, FIG. 2 is a schematic block diagram representation of an embodiment of the fluid infusion device 102. FIG. 2 depicts elements of the fluid infusion device 102 as functional blocks or modules, namely: a display element 202; a user interface 204; a memory 206; an electronics and processor module 208 (which may include or cooperate with one or more processor devices, controllers, state machines, or the like, to implement the controller 116 depicted in FIG. 1); a drive motor assembly 210; a force sensor 212; a power supply 214 such as a battery or a battery pack; and other infusion pump hardware, software, and applications 216. FIG. 2 also depicts the fluid reservoir 114 and the infusion set 104 in block format. The elements of the fluid infusion device 102 may be coupled together via an interconnection architecture 218 or arrangement that facilitates transfer of data, commands, power, etc.

The display element 202 represents the primary graphical interface of the fluid infusion device 102. The display element 202 may leverage any suitable display technology. The actual size, resolution, and operating specifications of the display element 202 can be selected to suit the needs of the particular application. Notably, the display element 202 may include or be realized as a touch screen display element that can accommodate touch screen techniques and technologies. In practice, the display element 202 may be driven by a suitable display driver to enable the fluid infusion device 102 to display physiological patient data, status information, clock information, alarms, alerts, and/or other information and data received or processed by the fluid infusion device 102.

The user interface 204 may include a variety of items such as, without limitation: a keypad, keys, buttons, a keyboard, switches, knobs (which may be rotary or push/rotary), a touchpad, a microphone suitably adapted to receive voice commands, a joystick, a pointing device, an alphanumeric character entry device or touch element, a trackball, a motion sensor, a lever, a slider bar, a virtual writing tablet, or any device, component, or function that enables the user to select options, input information, or otherwise control the operation of the fluid infusion device 102. In this context, the user interface 204 may cooperate with or include the display element 202 (if implemented as a touch screen). The user interface 204 allows a user to control the delivery of fluid via the infusion set 104.

The memory 206 may be realized as RAM memory, flash memory, EPROM memory, EEPROM memory, registers, a hard disk, a removable disk, a CD-ROM, or any other form of storage medium known in the art. In this regard, the memory 206 can be coupled to the electronics and processor module 208 such that the electronics and processor module 208 can read information from, and write information to, the memory 206. In the alternative, the memory 206 may be integral to the electronics and processor module 208. As an example, a processor device of the electronics and processor module 208 and the memory 206 may reside in an ASIC. In practice, a functional or logical module/component of the fluid infusion device 102 might be realized using program code that is maintained in the memory 206. Moreover, the memory 206 can be used to store data utilized to support the operation of the fluid infusion device 102, including, without limitation, glucose sensor data, sensor data generated by the bubble detector 106, force measurements, alert/alarm history, and the like (as will become apparent from the following description).

The electronics and processor module 208 may include or be implemented with a general purpose processor, a content addressable memory, a digital signal processor, an application specific integrated circuit, a field programmable gate array, any suitable programmable logic device, discrete gate or transistor logic, discrete hardware components, or any combination designed to perform the functions described here. A processor device may be realized as a microprocessor, a controller, a microcontroller, or a state machine. Moreover, a processor device may be implemented as a combination of computing devices, e.g., a combination of a digital signal processor and a microprocessor, a plurality of microprocessors, one or more microprocessors in conjunction with a digital signal processor core, or any other such configuration. As mentioned above, the controller 116 depicted in FIG. 1 can be realized with or implemented by the electronics and processor module 208. Moreover, a functional or logical module/component of the fluid infusion device 102 might be realized by, implemented with, and/or controlled by processing logic maintained by or included with the electronics and processor module 208. For example, the display element 202, the user interface 204, and/or the infusion pump hardware, software, and applications 216 (or portions thereof) may be implemented in or controlled by the electronics and processor module 208.

The drive motor assembly 124 includes an electric motor that is actuated and controlled by the electronics and processor module 208 to move an actuator, which in turn forces the medication fluid out of the fluid reservoir 114. For this particular embodiment, the force sensor 212 is operatively associated with the drive motor assembly 210. The force sensor 212 reacts to forces imparted thereto by the drive motor assembly 210, forces imparted to the drive motor assembly 210 via the fluid pressure of the fluid reservoir 114, and/or forces imparted to the drive motor assembly 210 via air pressure inside the housing of the fluid infusion device 102. The measured force can be used for various functions, safety checks, and operations that are unrelated to the disclosed bubble detection methodology.

The infusion pump hardware, software, and applications 216 are utilized to carry out fluid infusion features, operations, and functionality. Thus, the infusion pump hardware, software, and applications 216 may include or cooperate with the infusion set 104 and/or the fluid reservoir 114. In certain embodiments, the infusion pump hardware, software, and applications 216 includes a data communication module (e.g., a wireless radio or transceiver) that is compatible with at least one other device, system, or component. For example, a data communication module of the fluid infusion device 102 can be utilized to receive glucose sensor data from a continuous glucose monitor, to receive sensor data from the bubble detector 106, and/or to transmit data from the fluid infusion device 102 to a remote destination device. It should be appreciated that the infusion pump hardware, software, and applications 216 may leverage known techniques to carry out conventional infusion pump functions and operations, and such known aspects will not be described in detail here.

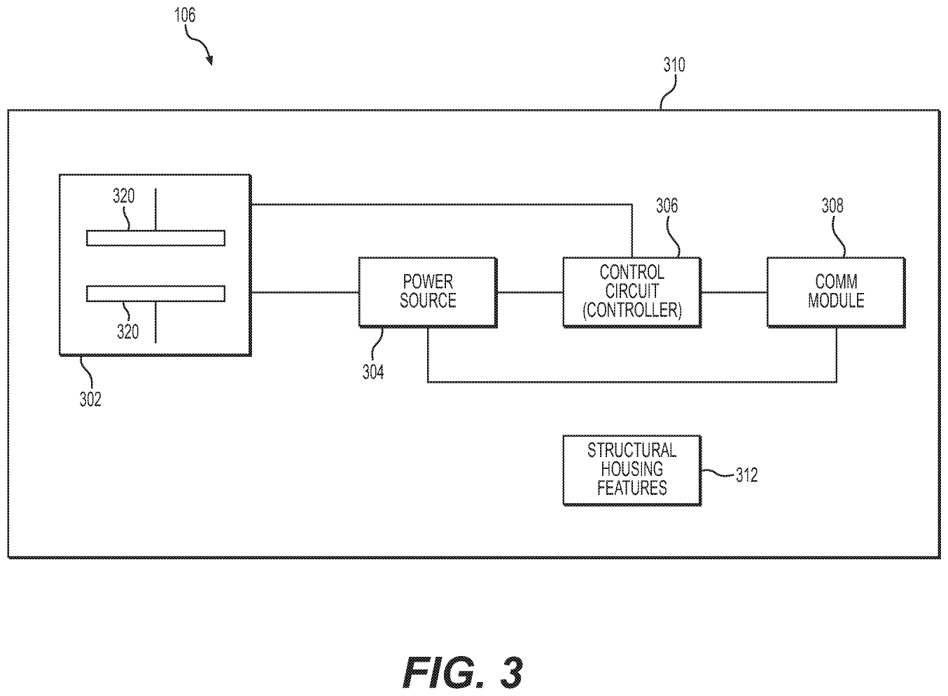

FIG. 3 is a schematic block diagram representation of an embodiment of the bubble detector 106 shown in FIG. 1. In practice, the bubble detector 106 can be fabricated and assembled into a variety of different shapes, sizes, and form factors. Regardless of the manner in which the bubble detector 106 is implemented, it is suitably configured and designed to detect changes in capacitance associated with the fluid delivery tube 108. The embodiment illustrated in FIG. 3 includes, without limitation: a capacitive sensor 302; a power source 304; a control circuit 306; and a communication module 308. These elements are coupled together or otherwise cooperate with each other to support the functions and detection features mentioned here. The components of the bubble detector 106 reside within a suitably sized and configured housing 310.

The bubble detector 106 is couplable to the fluid delivery tube 108 of the infusion set 104. In certain embodiments, the bubble detector 106 is removably attachable to the fluid delivery tube 108 such that the bubble detector 106 can be reused multiple times with replacement infusion sets 104. To this end, the housing 310 may include or cooperate with structural features 312 that are designed, configured, and fabricated in an appropriate manner to secure the bubble detector 106 to the fluid delivery tube 108, and to maintain the capacitive sensor 302 in position relative to the fluid delivery tube 108.

The capacitive sensor 302 includes capacitor electrodes 320, which are electrically connected to the power source 304. Although the power source 304 is depicted as an internal component, in alternate embodiments it may be an external component that is electrically connected to the internal components. The power source 304 can be a rechargeable battery (for a durable and reusable embodiment of the bubble detector 106) or a single-use battery (for a disposable embodiment of the bubble detector 106). The power source 304 is suitably configured and controlled to provide the desired operating voltage across the capacitor electrodes 320. The power source 304 can also provide the necessary operating power to the control circuit 306, the communication module 308, and/or other components of the bubble detector 106. In this regard, the power source 304 can be controlled to generate any desired voltage potential, a transient voltage pattern, a voltage waveform, a swept voltage, or the like. The voltage applied to the capacitor electrodes 320 enables the bubble detector 106 to monitor, sense, or otherwise measure a detectable characteristic that is associated with the capacitance of the capacitive sensor 302.

Although not shown in FIG. 3, when the bubble detector 106 is installed onto the fluid delivery tube 108, the tube 108 resides between the two electrodes 320. Accordingly, the dielectric “material” of the capacitive sensor 302 is the combination of the material used to fabricate the tube 108 and the fluid (or other contents) that resides within the tube 108. Conceptually, the power source 304 is controlled or regulated in a particular manner that allows monitoring of changes in capacitance that are caused by changes in the dielectric “material” that resides between the electrodes 320. For example, if the tube 108 is empty (no fluid is present), then the dielectric constant of the capacitive sensor 302 will be a first value. If the tube 108 is full of bubble-free medication fluid, then the dielectric constant of the capacitive sensor 302 will be a second value that is different than the first value. If, however, the tube 108 contains the same medication fluid with bubbles present, then the dielectric constant of the capacitive sensor 302 will be a third value that is different than the first and second values. Since an empty tube would yield a first capacitance value, and a bubble-free fluid would yield a second capacitance value, any presence of bubbles in the fluid would yield a capacitance value between the first and the second. The presence of bubbles changes the effective dielectric constant of the fluid and causes changes in the overall capacitance value. Changes to the dielectric constant result in corresponding changes to the capacitance of the capacitive sensor 302, and the medical device system 100 monitors the output of the bubble detector 106 to detect such changes.

The control circuit 306 may be realized as a suitably arranged electrical circuit that controls the operation of the capacitive sensor 302 to measure the capacitance associated with the fluid present in the fluid delivery tube 108. Although depicted as a separate block, the control circuit 306 may include the power source 304 and/or the communication module 308. The control circuit 306 regulates the voltage applied across the electrodes 320 for purposes of checking the capacitance of the capacitive sensor 302 in an ongoing manner. The control circuit 306 may also serve as an interface circuit to translate the response of the capacitive sensor 302 into usable sensor data that can be analyzed to determine whether bubbles are present in the tube 108. To this end, the control circuit 306 may include an analog-to-digital converter, a digital encoder, and/or other circuit elements to convert “raw” voltage or current into sensor data that is indicative of the measured capacitance associated with the presence of bubbles in the fluid in the tube 108. The sensor data can indicate instantaneous capacitance values, a transient response, capacitance values over time, or the like.

In certain embodiments, the communication module 308 is suitably configured to communicate the sensor data (and other information if so desired) from the bubble detector to a destination device, component, or system. In alternative embodiments, the communication module 308 communicates notifications, alerts, or status information from the bubble detector to a destination device, component, or system. For this particular example, the communication module 308 wirelessly communicates with the fluid infusion device 102. Accordingly, the communication module 308 includes or cooperates with a wireless radio or transceiver that is compatible with the fluid infusion device 102. This allows the bubble detector 106 to transmit appropriately formatted or processed sensor data (obtained from the capacitive sensor 302) to the fluid infusion device 102 for review, analysis, and further processing as needed.

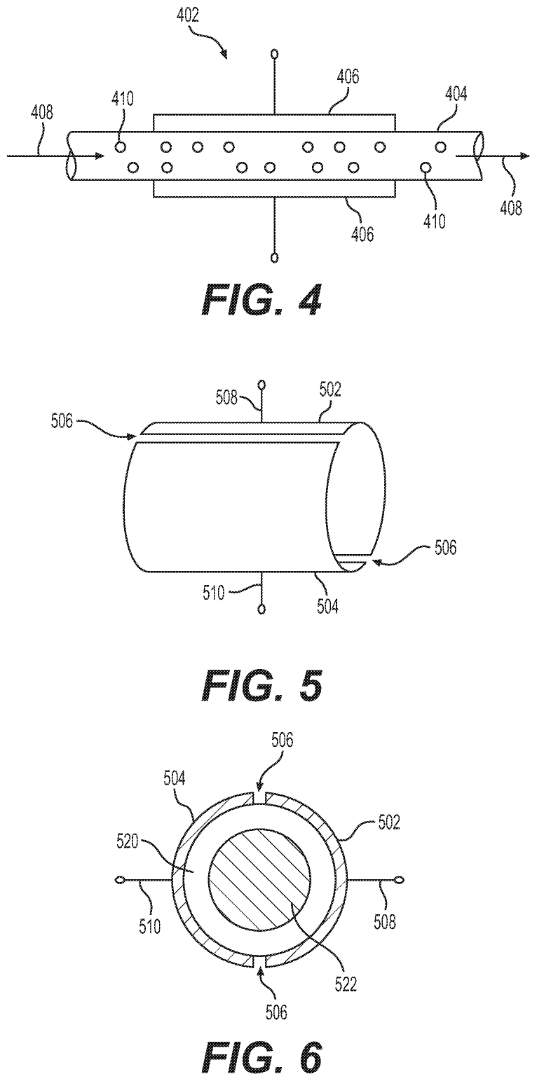

As mentioned previously, the bubble detector 106 can be removably attached to the fluid delivery tube 108 in such a way that the fluid delivery tube 108 is securely held in position between the electrodes 320 of the capacitive sensor 302. More specifically, the housing 310 is couplable to the tube 108 in a particular manner to hold the capacitor electrodes 320 in physical contact with the outer surface of the tube 108 (as schematically shown in FIG. 4 and FIG. 6). The physical contact between the electrodes 320 and the tube 108 ensures that the tube 108 and its contents can reliably serve as the dielectric material of the capacitive sensor 302.

The structural features 312 of the housing 310 may be realized in various ways to suit the needs of the particular form factor of the bubble detector 106. For example, the housing 310 may be configured as a hinged “clamshell” with structural features 312 that are operable to clamp the housing 310 around the fluid delivery tube 108. As another example, the housing 310 may be fabricated as a two-part assembly that snaps together around the tube 108. In this regard, the structural features 312 may include or be realized as a latch, a snap-fit element, notches, a hinge, a press-fit element, or other engagement mechanisms that are operable to removably attach the housing 310 to the tube 108.

FIG. 4 is a schematic side view of a capacitive sensor 402 and a fluid delivery tube 404. The capacitive sensor 402 includes two electrodes 406 surrounding the tube 404. The electrodes 406 are in physical contact with the outer surface of the tube 404, such that the tube 404 and the fluid 408 within the tube together represent the dielectric material of the capacitive sensor 402. FIG. 4 depicts the fluid 408 flowing through the tube 404 from left to right, wherein bubbles 410 are present in the fluid 408. As explained above, the presence of bubble-free fluid (e.g., insulin) between the electrodes 406 results in one detectable capacitance, while the presence bubbles within the fluid 408 results in a different and distinguishable capacitance.

Although the capacitive sensor can be implemented in various ways, certain embodiments utilize semi-cylindrical electrodes, as depicted in FIG. 5. The electrodes 502, 504 shown in FIG. 5 are semi-cylindrical in shape, and they are separated by gaps 506. The electrode 502 is electrically connected to a first lead 508, and the electrode 504 is electrically connected to a second lead 510. Notably, the semi-cylindrical shape and size of the electrodes 502, 504 are selected to match the shape and size of the fluid infusion tube that will be located between the electrodes 502, 504. In this regard, FIG. 6 is a cross sectional view that depicts the electrodes 502, 504 surrounding a fluid delivery tube 520. In FIG. 6, the tube 520 contains medication fluid 522 to be delivered to a user. FIG. 6 illustrates how the semi-cylindrical electrodes 502, 504 conform to the outer surface of the tube 520. An implementation of a semi-cylindrical capacitive sensor and related operating methodologies are disclosed in A Semicylindrical Capacitive Sensor With Interface Circuit Used For Flow Rate Measurement, by Chiang et al., IEEE Sensors Journal, Vol. 6, No. 6, December 2006. The relevant content of the Chiang article is incorporated by reference herein. In certain embodiments, the bubble detector 106 disclosed here can also leverage the flow rate measurement technique disclosed in the Chiang article, such that the detected capacitive sensor data can be used for both flow rate detection and bubble detection.

In accordance with a typical use case, the bubble detector 106 is installed onto the fluid infusion tube 108 at a downstream location close to the infusion site component 112 (see FIG. 1). The installation can be performed before or after the infusion set 104 has been secured to the body of the patient; the installation can be performed before of after the infusion set 104 has been connected to the fluid infusion device 102. After the tube 108 has been primed with the medication fluid, the bubble detector 106 can be operated to monitor the capacitance of the tube 108 and the fluid passing through the tube 108 in an ongoing manner.

An exemplary methodology for detecting the presence of one or more bubbles in the tube 108 may begin by receiving sensor data from the capacitive sensor 302, wherein the sensor data is indicative of measured capacitance associated with the fluid in the tube 108. In certain embodiments, the sensor data is transmitted from the communication module 308 of the bubble detector 106 to a suitably configured controller that resides at the fluid infusion device 102. The controller functions to receive and analyze the sensor data from the bubble detector, and to determine the presence of one or more bubbles in the fluid delivery tube 108 based on an analysis or review of the received sensor data. In alternative embodiments, the controller is realized as an integral component of the bubble detector 106 itself. In such alternative embodiments, the sensor data need not be transmitted or communicated (although the data might be routed internally from one component or module to another). Instead, the sensor data can be provided to or accessed by the integrated controller for review and analysis.

The sensor data received from the capacitive sensor is analyzed in an appropriate manner to determine whether or not the fluid in the tube 108 is free of bubbles. For example, when the analyzed sensor data indicates a measured capacitance having a first detectable characteristic, the controller determines that a bubble-free state of the fluid exists. In contrast, when the analyzed sensor data indicates a measured capacitance having a second detectable characteristic that is distinguishable from the first detectable characteristic, then the controller determines that one or more bubbles are present in the fluid delivery tube 108. Therefore, if the fluid remains bubble-free, then the capacitance of the capacitive sensor will remain substantially constant and within a range of predicable values during normal operation of the fluid infusion device. If the fluid contains a detectable bubble, then the capacitance of the sensor will immediately change when the bubble flows to a location between the electrodes of the capacitive sensor. If the bubble flows past the electrodes, then the measured capacitance will return to the nominal or expected value. The controller can detect the sudden change in capacitance and take appropriate action. As another example, if the fluid contains a concentration of micro-bubbles, then the capacitance of the sensor will change from the normally expected value to a different and readily distinguishable value. The onset of the change may be sudden or gradual. The controller can also detect a persistent (or somewhat persistent) change in capacitance, which may indicate the presence of micro-bubbles in the fluid.

If the controller determines that one or more bubbles are present in the fluid delivery tube 108, then it can initiate one or more corrective actions. A corrective action can be executed at the medication fluid infusion device, at the bubble detector, at a client device owned or operated by the patient, at a client device owned or operated by a caregiver, or the like. In practice, the corrective action may include any of the following, without limitation: generating a notification; generating an alert; generating an alarm; generating a warning; sending a message; modifying the fluid delivery operation of the fluid infusion device; temporarily halting fluid delivery; calling a telephone number of the patient or a caregiver; and/or adjusting therapy-related settings of the fluid infusion device.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A medical device system comprising:
a medication fluid infusion device comprising a fluid reservoir, the medication fluid infusion device operable to regulate and control delivery of a medication fluid from the fluid reservoir;
an infusion set comprising a fluid delivery tube and an infusion site component fluidly and mechanically coupled to a downstream end of the fluid delivery tube, the infusion set couplable to the medication fluid infusion device to establish a fluid connection with the fluid reservoir;
a bubble detector couplable to the fluid delivery tube of the infusion set, the bubble detector comprising:
a capacitive sensor;
a power source to provide operating voltage to the capacitive sensor;
a control circuit to control operation of the capacitive sensor to measure capacitance associated with fluid in the fluid delivery tube; and
a communication module to communicate sensor data from the bubble detector, the sensor data indicative of the measured capacitance associated with the fluid in the fluid delivery tube; and
a controller to receive and analyze the sensor data from the bubble detector, and to determine presence of one or more bubbles in the fluid delivery tube based on analysis of the sensor data.

2. The medical device system of claim 1, wherein the medication fluid infusion device comprises the controller as an integral component.

3. The medical device system of claim 2, wherein the communication module wirelessly communicates the sensor data to the medication fluid infusion device.

4. The medical device system of claim 1, wherein the bubble detector comprises the controller as an integral component.

5. The medical device system of claim 1, wherein:
the bubble detector comprises a housing for at least the capacitive sensor; and
the housing comprises structural features to secure the bubble detector to the fluid delivery tube and to maintain the capacitive sensor in position relative to the fluid delivery tube.

6. The medical device system of claim 5, wherein the structural features are operable to clamp the housing around the fluid delivery tube.

7. The medical device system of claim 5, wherein the structural features are operable to removably attach the housing to the fluid delivery tube.

8. The medical device system of claim 1, wherein the controller is operative to:
determine a bubble-free state of the fluid in the fluid delivery tube when the sensor data indicates measured capacitance having a first detectable characteristic; and
determine presence of one or more bubbles in the fluid delivery tube when the sensor data indicates measured capacitance having a second detectable characteristic that is distinguishable from the first detectable characteristic.

9. The medical device system of claim 1, wherein the medication fluid infusion device is an insulin infusion pump, the medication fluid is insulin, and the infusion set is a patient-worn insulin infusion set.

10. The medical device system of claim 1, wherein the capacitive sensor comprises semi-cylindrical electrodes.

11. The medical device system of claim 10, wherein:
the fluid delivery tube has an outer surface; and
the bubble detector is couplable to the fluid delivery tube to hold the semi-cylindrical electrodes in contact with the outer surface of the fluid delivery tube.

12. The medical device system of claim 1, wherein the controller initiates a corrective action at the medication fluid infusion device in response to a determination of the presence of one or more bubbles in the fluid delivery tube.

13. A bubble detector for detecting presence of one or more bubbles in a fluid delivery tube, the bubble detector comprising:
a capacitive sensor comprising capacitor electrodes;
a control circuit to control operation of the capacitive sensor to measure capacitance associated with fluid in the fluid delivery tube;
a power source to provide operating voltage across the capacitor electrodes and to provide operating power to the control circuit;
a communication module to communicate sensor data from the bubble detector, the sensor data indicative of the measured capacitance associated with the fluid in the fluid delivery tube; and
a housing for at least the capacitive sensor, the housing comprising structural features to secure the bubble detector to the fluid delivery tube and to maintain the capacitor electrodes in position relative to the fluid delivery tube.

14. The bubble detector of claim 13, further comprising a controller operable to determine presence of one or more bubbles in the fluid delivery tube based on analysis of the sensor data.

15. The bubble detector of claim 13, wherein the communication module wirelessly communicates the sensor data to a medication fluid infusion device that regulates and controls delivery of a medication fluid through the fluid delivery tube.

16. The bubble detector of claim 13, wherein the structural features are operable to removably clamp the housing around the fluid delivery tube.

17. The bubble detector of claim 13, wherein:
the fluid delivery tube has an outer surface; and
the housing is couplable to the fluid delivery tube to hold the capacitor electrodes in contact with the outer surface of the fluid delivery tube.

18. A method of detecting presence of one or more bubbles in a fluid delivery tube, the method comprising the steps of:
receiving sensor data from a capacitive sensor coupled to the fluid delivery tube, the sensor data indicative of measured capacitance associated with the fluid in the fluid delivery tube;
analyzing the sensor data received from the capacitive sensor;
determining a bubble-free state of the fluid in the fluid delivery tube when the analyzed sensor data indicates measured capacitance having a first detectable characteristic;
determining presence of one or more bubbles in the fluid delivery tube when the analyzed sensor data indicates measured capacitance having a second detectable characteristic that is distinguishable from the first detectable characteristic; and
initiating a corrective action in response to determining the presence of one or more bubbles in the fluid delivery tube.

19. The method of claim 18, wherein:
the fluid delivery tube is integrated with an infusion set of a medication fluid infusion device; and
the corrective action comprises generating a notification, an alert, or a warning at the medication fluid infusion device.

20. The method of claim 18, wherein:
the fluid delivery tube is integrated with an infusion set of a medication fluid infusion device; and
the corrective action comprises modifying a fluid delivery operation of the medication fluid infusion device.
